SEQUENCE LISTING (5 sequences)

SEQ ID NO: 1 - protein, 19 aa, Human papillomavirus type 16
DSTLRLCVQSTHVDIRTLE

SEQ ID NO: 2 - DNA, 24 bp, Human papillomavirus type 16
cgtaaacgttttccctattttttt

SEQ ID NO: 3 - DNA, 21 bp, Human papillomavirus type 16
taccctaaatactctgtattg

SEQ ID NO: 4 - DNA, 21 bp, Human papillomavirus
taccctaaataccctatattg

SEQ ID NO: 5 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

(19) United States
(12) Patent Application Publication
(10) Pub. No.: US 2004/0151723 A1
(43) Pub. Date: Aug. 5, 2004

(54) Novel epitope of human papilloma virus e7 antigen and cd4-positive t cells activated by the epitope

(76) Inventors: Hiroo Maeda, Saitama-Ken (JP); Mitsuo Okubo, Saitama-Ken (JP)

Correspondence Address: FOLEY AND LARDNER, SUITE 500, 3000 K STREET NW, WASHINGTON, DC, 20007, US

(21) Appl. No.: 10/479,541

(22) PCT Filed: Jun. 10, 2002
(86) PCT No.: PCT/JP02/05747

(30) Foreign Application Priority Data
Jun. 8, 2001 (JP) 2001-173803

(51) Int. Cl. A61K 39/395; C07H 21/04; C12N 5/08; C07K 14/74

(52) U.S. Cl. 424/155.1; 530/350; 435/372; 435/69.1; 435/320.1; 536/23.5

(57) ABSTRACT

An object of the present invention is to provide an epitope on a human papillomavirus antigen, which activates a CD4-positive T cell of a uterine cancer patient, and a CD4-positive T cell specific to cancerous and precancerous lesions of the uterus, which is activated by this epitope. An epitope according to the present invention comprises (a) the amino acid sequence of SEQ ID NO: 1, or (b) a modified amino acid sequence of the amino acid sequence of SEQ ID NO: 1 that has one or more modifications selected from the group consisting of a substitution, a deletion, an addition and an insertion and can activate CD4-positive T cells specific to cancerous and precancerous lesions of the uterus.

### BACKGROUND OF THE INVENTION

1. Field of the Invention

The present invention relates to epitopes of human papillomavirus E7 antigens, and more specifically to E7 antigen epitopes, which activate the ability of CD4-positive T cells to induce tumor cytotoxicity and to induce the production of an antibody specific to uterine cancer. Further, the present invention relates to the treatment and prevention of uterine cancer using CD4-positive T cells activated by epitopes of human papillomavirus E7 antigens.

2. Background Technology

Among so-called immunity system cells, T cells have important functions, for example, in eliminating foreign antigens to protect the body from infections or in exhibiting cytotoxicity to remove cancerous cells. These T cells accurately recognize amino acid sequences of antigenic peptides to activate T cells themselves. The structure that recognizes an antigenic peptide is called a T cell receptor, and the specific amino acid sequence binding to such T cell receptor is called “an antigen determinant: epitope.” Since a T cell dependent-immune reaction cannot start unless a T cell recognizes this epitope, an epitope is considered to be the most important part of an antigen. Accordingly, it is to become the primary objective to determine an epitope of an antigen in research aiming to treat and control tumors, infections or the like.

In order to immunologically remove a tumor, among immune system cells, an antigen-presenting cell, a T cell and an antigen that binds to both cells (referred to as a tumor rejection antigen to distinguish from an antigen which causes cancer) have to be identified. Practically, it is difficult to identify a tumor rejection antigen, which is recognized by a cytotoxic T-lymphocyte (referred to as CTL hereinafter) in a patient although analyses in animal experiments have been reported. A possible reason is, for example, that in a cancer patient, the expression of a class I molecule of major histocompatibility complex (referred to as MHC hereinafter) binding to a cancer antigen along with a CTL has already been weakened in a cancerous cell and the tolerance to a tumor rejection antigen has been established in the CTL. Thus, the establishment of tolerance is recognized in cellular immunity in the body with progressed cancer. However, on the other hand, humoral immunity is maintained and keeps producing antibodies against cancer antigens. This indicates that a CD4-positive T cell maintains its mechanism to recognize an antigen represented on an MHC class II molecule.

Now, human papillomavirus (referred to as HPV hereinafter) causes persistent infection in the basalis of the cervical epithelium. An observation of surgical specimens of cervical cancer patients reported that HPV viruses were detected from 80% of cancerous lesions. Further, HPV virus DNAs were detected at a frequency of 50% or more also in precancerous lesions in the cervix. Further, it is known from a study using DNA of this virus that E6 and E7 proteins produced by this virus transform fibroblasts derived from the human uterus and suppress the action of p53, a cancer-suppressing gene product. Thus, HPV is known to be one of the causative agents for uterine cancer (zur Hausen H. Human papillomaviruses in the pathogenesis of anogenital cancer. Virology 1991, 184:9-13).

Although the treatment of cancerous cells with CTL and NK cells using HPV-derived antigens, the decrement in MHC class I expression and the tolerance of T cells have been obstacles (Hilders C G, Munoz I M, Nooyen Y, Fleuren G J. Altered HLA expression by metastatic cervical carcinoma cells as a factor in impaired immune surveillance. Gynecol Oncol. 1995, 57:366-375; 16. Kono K, Ressing M E, Brandt R M, Melief C J, Potkul R K, Andersson B, Petersson M, Kast W M, Kiessling R. Decreased expression of signal-transducing zeta chain in peripheral T cells and natural killer cells in patients with cervical cancer. Clin Cancer Res. 1996, 2:1825-1828). On the other hand, it has been believed that CD4-positive helper T (Th) cells function only for antibody production although they establish no tolerance. Recently, it has been revealed that Th cells also have cytotoxic activity, such as TNFα and Fas ligand, owing to progress in analyses using animal experiments or the like. Further, it has also been revealed that a Th1-type T cell, a member of Th cells, expresses TNFα more strongly than a Th2-type T cell, and exhibits synergistic effect in the activation of an antigen-presenting cell and CTL and the activation of the Th1-type T cell, via cytokines such as IFNγ and IL-12. Accordingly, expectation for therapeutic effect on tumor cells is shifting from MHC class I-restricted CD8-positive T cells to MHC class II-restricted CD4-positive T cells (Yasukawa M, Ohminami H, Kaneko S, Yakushijin Y. Nishimura Y, Inokuchi K, Miyakuni T, Nakao S, Kishi K, Kubonishi I, Dan K, Fujita S. CD4(+) cytotoxic T cell clones specific for bcr-abl b3a2 fusion peptide augment colony formation by chronic myelogenous leukemia cells in a b3a2-specific and HLA-DR-restricted manner. Blood 1998, 92:3355-3361).

Under these circumstances, as for some MHC molecules, amino acid sequences that are presumed to bind to the MHC molecules were analyzed using molecular biological physical procedures, and their consensus motifs have been reported (Hammer J, Valsasnini P, Tolba K, Bolin D, Higelin J, Takacs B, Sinigaglia F. Promiscus and allele-specific anchors in HLA-DR-binding peptides. Cell 1993, 74:197-203; Fujisao S, Matsushita S, Nishi T, Nishimura Y. Identification of HLA-DR9 (DRB1*0901)-binding peptide motifs using a phage fUSE5 random peptide library. Human Immunol. 1996, 45:131-136). However, as for HPV, an epitope of tumor rejection antigen which recognizes MHC class II-restricted CD4-positive cells has not been so far identified.

### SUMMARY OF THE INVENTION

The present inventors found a novel sequence, which can activate a human Th2-type T cell (Th2 cell) in vitro in a human papillomavirus E7 antigen, one of the causes of uterine cancer.

Also, the present inventors synthesized a peptide based on this specified amino acid sequence and succeeded in inducing a human Th1-type T cell (Th1 cell), which can induce tumor cell cytotoxicity, by stimulating patient's peripheral blood mononuclear cells in vitro using this peptide and IL-12. This T cell produces IFNγ, TNFα and Fas ligands, and T cells producing these factors are effective cells for cellular therapy against cancerous and precancerous lesions of the uterus in which human papillomaviruses cause persistent infection, expressing E7 proteins.

An object of the present invention is to provide an epitope on a human papillomavirus antigen, which activates a CD4-positive T cell of a uterine cancer patient.

An object of the present invention is to provide a CD4-positive T cell specific to cancerous and precancerous lesions of the uterus and a method for producing the same, a pharmaceutical composition for the therapy and prevention of uterine cancer for use in tumor therapy, and a composition for activating the ability to induce tumor cytotoxicity and antibody production of the CD4-positive T cell.

Further, an epitope of human papillomavirus antigen according to the present invention comprises the following amino acid sequence:

(a) an amino acid sequence of SEQ ID NO: 1, or

(b) a modified amino acid sequence of SEQ ID NO: 1, which has one or more modifications selected from the group consisting of a substitution, a deletion, an addition, and an insertion and can activate a CD4-positive T cell specific to cancerous and precancerous lesions of the uterus.

A CD4-positive Th1 cell specific to cancerous and precancerous lesions of the uterus according to the present invention is obtainable by culturing peripheral blood mononuclear cells in a medium containing an epitope according to the present invention and IL-12.

A CD4-positive Th2 cell specific to cancerous and precancerous lesions of the uterus according to the present invention is obtainable by culturing peripheral blood mononuclear cells in a medium containing an epitope according to the present invention.

### BRIEF DESCRIPTION OF THE DRAWINGS

FIG. 1 shows the result of two-color flow cytometry in the presence of stimulation with HPV E7-5.1 antigen (left) and HPV E7-4.1 antigen (right) in Example 4. The vertical axis represents relative fluorescence intensity of CD4 and the horizontal axis represents relative fluorescence intensity of IL-4. The numbers in the Figure show the frequency of IL-4-producing CD4-positive Th2 cells specific to HPV antigen epitopes.

FIG. 2 shows the correlation between the frequency of CD4-positive cases and the concentration of HPV E7-4.1 peptide antigen in Example 4.

FIG. 3 shows the effect of HPV E7-4.1 antigen stimulation on TNFα production of CD4-positive T cells in the presence of IL-2 and IL-12 in Example 5. The results are without the antigen stimulation (left) and with the antigen stimulation (right). The vertical axis represents relative fluorescence intensity of CD4 and the horizontal axis represents relative fluorescence intensity of TNFα. The numbers in the Figure show the frequency of TNFα-producing CD4-positive T cells specific to the HPV antigen epitope.

FIG. 4 shows the expression of Fas ligand after HPV E7-4.1 antigen stimulation in the presence of IL-12 in Example 5. The results are without antigen stimulation (left) and with antigen stimulation (right). The vertical axis represents relative fluorescence intensity of Fas ligand. The numbers in the Figure show the frequency of Fas ligand-positive T cells.

### DETAILED DESCRIPTION OF THE INVENTION

### E7 Antigen Epitope

An E7 epitope according to the present invention comprises the amino acid sequence of SEQ ID NO: 1 or a modified sequence thereof. The number of modifications in the modified sequence can be one to several.

In the present invention, “amino acid sequence that can activate CD4-positive T cells specific to cancerous and precancerous lesions of the uterus” means an amino acid sequence which is evaluated to be able to activate CD4-positive T cells specific to cancerous and precancerous lesions of the uterus by those skilled in the art; for example, it means an amino acid sequence which permits the growth of CD4-positive Th2 cells that can induce production of an antibody against cancerous and precancerous lesions of the uterus, in which an E7 protein of human papillomavirus 16 is expressed, under the same conditions as described in Example 4, and an amino acid sequence which permits the growth of CD4-positive Th1 cells that can induce tumor cytotoxicity against cancerous and precancerous lesions of the uterus, in which E7 protein of human papillomavirus 16 is expressed, under the same conditions as described in Example 5.

The term “cancer of the uterus” in the present invention is intended to mean uterine cancer and cervical cancer.

The term “cancerous and precancerous lesions of the uterus” in the present invention refers to those recognized as cancerous and precancerous lesions of the uterus with the naked eye or by cytodiagnosis or biopsy. The cancerous and precancerous lesions of the uterus can be those caused by papillomavirus, in particular, by human papillomavirus 16.

Examples of “cancerous lesions of the uterus” include carcinoma in situ (CIS) and intraepithelial carcinoma (IC).

Examples of “precancerous lesions of the uterus” include cases with a low level of deformity of lesions (CIN1, CIN2) and a precancerous state (CIN3).

“Papillomavirus” in the present invention can be human papillomavirus and more specifically, human papillomavirus 16, human papillomavirus 18, and human papillomavirus 33 (Yoshikawa H, Kawana T, Kitagawa K, Mizuno M, Yoshikura H, Iwamoto A. Detection and typing of multiple genital human papillomaviruses by DNA amplification with consensus primers. Jpn J Cancer Res, 1991, 82:524-531; Nakagawa S, Yoshikawa H, Onda T, Kawana T, Iwamoto A, Taketani Y. Type of human papillomavirus is related to clinical features of cervical carcinoma. Cancer 1996, 78:1935-1941).

### Activation of CD4-positive T Cell

An epitope according to the present invention can not only activate the ability of a CD4-positive Th2 cell to induce antibody production induction (Example 4) but also activate the ability of a CD4-positive Th1 cell, in which immunological tolerance is established, to induce tumor cytotoxicity in the presence of IL-12 (Example 5).

Therefore, according to the present invention, there is provided a CD4-positive Th1 cell specific to cancerous and precancerous lesions of the uterus, which is obtainable by culturing peripheral blood mononuclear cells in a medium comprising an epitope according to the present invention and IL-12.

Also, according to the present invention, there is provided a CD4-positive Th2 cell specific to cancerous and precancerous lesions of the uterus, which is obtainable by culturing peripheral blood mononuclear cells in a medium comprising an epitope according to the present invention.

According to the present invention, there are provided an agent for activating the ability of a CD4-positive Th1 cell to induce tumor cytotoxicity specific to cancerous and precancerous lesions of the uterus, comprising an epitope according to the present invention, and an agent for activating the ability of a CD4-positive Th2 cell to induce the production of an antibody specific to cancerous and precancerous lesions of the uterus, comprising an epitope according to the present invention.

Also, according to the present invention, there are provided use of an epitope according to the present invention for the production of an agent for activating the ability of a CD4-positive Th1 cell to induce tumor cytotoxicity specific to cancerous and precancerous lesions of the uterus and use of an epitope according to the present invention for the production of an agent for activating the ability of a CD4-positive Th2 cell to induce the production of an antibody specific to cancerous and precancerous lesions of the uterus.

Further, according to the present invention, there are provided a method for activating the ability of a CD4-positive Th1 cell to induce tumor cytotoxicity specific to cancerous and precancerous lesions of the uterus, comprising the step of bringing an epitope according to the present invention into contact with the CD4-positive Th1 cell in the presence of IL-12, and a method of activating the ability of a CD4-positive Th2 cell to induce the production of an antibody specific to cancerous and precancerous lesions of the uterus, comprising the step of bringing an epitope according to the present invention into contact with the CD4-positive Th2 cell.

Whether T cells are CD4-positive or not can be determined by detecting a cell-surface marker of the T cells, a CD4 molecule, and cytokine or lymphokine in combination (Openshaw P, Murphy E E, Hosken N A, Maino V, Davis K, Murphy K, O'Garra A. Heterogeneity of intracellular cytokine synthesis at the single-cell level in polarized T helper 1 and T helper 2 populations. J Exp Med. 1995, 182:1357-1367).

A CD4-positive Th1 cell and a CD4-positive Th2 cell according to the present invention can specifically recognize E7 proteins of human papillomavirus 16, in particular a peptide comprising the amino acid sequence of SEQ ID NO: 1 (Example 4). Although not restricted by the following theory, an epitope according to the present invention binding to an MHC class II molecule is presented on an antigen-presenting cell, and the Th1 cell and the Th2 cell are activated by the presented epitope.

Accordingly, a CD4-positive Th1 cell according to the present invention can induce tumor cytotoxicity against cancerous and precancerous lesions of the uterus. The term “tumor cytotoxicity” refers not only to tumor cytotoxicity by cytotoxic factors but also to tumor cytotoxicity by cytotoxic cells such as natural killer cells. The induction of tumor cytotoxicity can be confirmed, for example, by the production of TNFα or Fas ligand as described in Example 5.

Further, a CD4-positive Th2 cell according to the present invention can induce the production of an antibody specific to cancerous and precancerous lesions of the uterus. The induction of the specific antibody production can be confirmed, for example, as described in Example 4.

A CD4-positive Th1 cell according to the present invention can be produced by culturing peripheral blood mononuclear cells isolated from the blood of a uterine cancer patient or a person suspected to be a uterine cancer patient in a medium comprising an epitope according to the present invention and IL-12, and recovering the CD4-positive Th1 cells (see Example 4).

Further, a CD4-positive Th2 cell according to the present invention can be produced by culturing peripheral blood mononuclear cells isolated from the blood of a uterine cancer patient or a person suspected to be a uterine cancer patient in a medium comprising an epitope according to the present invention, and recovering CD4-positive Th2 cells (see Example 5).

The medium to be used can be a medium generally used for culturing peripheral blood mononuclear cells, such as an RPMI medium and a Dulbecco's modified Eagle's medium (DME). If necessary, antibiotics, cytokines or the like can be added to a medium.

An epitope according to the present invention can be added to a medium at a final concentration of 10 to 50 nmol.

In the production of CD4-positive Th1 cells, IL-12 is added in addition to an epitope according to the present invention. The IL-12 can be added to a medium, for example, at a final concentration of 0.2 ng/ml.

Examples of IL-12 to be used in the present invention include an IL-12 isolated from the body of humans, mice, or the like, a recombinant IL-12, and those having biological functions equivalent to these IL-12; those commercially available can be used. A recombinant IL-12 can be produced by expressing a corresponding human IL-12 gene (e.g., Wolf S. F. et al., J. Immunol., 146, p3074 (1991)) or a mouse IL-12 gene (e.g., Schoenhaut D. S. et al., J. Immunol., 148, pp3433-3440 (1992)) in an appropriate host cell.

Cultivation can be carried out under the conditions generally used for the cultivation of peripheral blood mononuclear cells, for example, at about 37° C. for 12 to 60 hours.

In the present invention, “peripheral blood mononuclear cells” can be isolated from the blood of a uterine cancer patient or a person suspected to be a uterine cancer patient.

In the present invention, the term “uterine cancer patient” refers to a patient who is confirmed to have cancerous or precancerous lesions of the uterus in the uterus or the cervix of uterus with the naked eye, or by cytodiagnosis or by biopsy.

In the present invention, “a person suspected to be a uterine cancer patient” refers to, for example, a person in which cancerous and precancerous lesions of the uterus are not observed by diagnosis, but infection with human papillomavirus, specifically human papillomavirus 16, is conformed in a tissue taken from the uterus or the cervix. Whether infected with human papillomaviruses can be determined, for example, by the method described in Example 1.

CD4-positive Th1 cells that can induce tumor cytotoxicity can be selected and recovered using the production of INFγ, TNFα, and/or Fas ligand as an index. CD4-positive Th2 cells that can induce specific antibody production can be selected and recovered using the production of INFγ as an index. In both cases, the selection and recovery can be carried out by flow cytometry.

A specific example of a CD4-positive Th1 cell according to the present invention is a CD4-positive Th1 cell that can induce the tumor cytotoxicity against cancerous and precancerous lesions of the uterus in which E7 proteins of human papillomavirus are being expressed, which is obtainable by culturing peripheral blood mononuclear cells isolated from the blood of a uterine cancer patient or a person suspected to be a uterine cancer patient in a medium comprising a peptide comprising the amino acid sequence of SEQ ID NO: 1 and IL-12.

A specific example of a CD4-positive Th1 cell according to the present invention is a CD4-positive Th1 cell that can induce the production of an antibody specific to cancerous and precancerous lesions of the uterus in which E7 proteins of human papillomavirus are being expressed, which is obtainable by culturing peripheral blood mononuclear cells isolated from the blood of a uterine cancer patient or a person suspected to be a uterine cancer patient in a medium comprising a peptide comprising the amino acid sequence of SEQ ID NO: 1.

### Tumor Therapy by Activated CD4-positive T Cells

A CD4-positive Th1 cell and a CD4-positive Th2 cell according to the present invention can be used for the treatment and prevention of uterine cancer since they induce tumor cytotoxicity and the production of antibody specific to tumors, respectively. More specifically, uterine cancer can be treated and prevented by returning the CD4-positive Th1 cells and CD4-positive Th2 cells according to the present invention into the patient's body.

Therefore, according to the present invention, there are provided a pharmaceutical composition for the treatment and prevention of uterine cancer comprising a CD4-positive Th1 cell according to the present invention, and use of a CD4-positive Th1 cell according to the present invention for producing a pharmaceutical for use in the treatment and prevention of uterine cancer.

Also, according to the present invention, there is provided a method for treating and preventing uterine cancer, comprising the steps of culturing peripheral blood mononuclear cells isolated from the blood of a uterine cancer patient or a person suspected to be a uterine cancer patient in a medium comprising an epitope according to the present invention and IL-12, recovering CD4-positive Th1 cells from the culture, and then returning the CD4-positive Th1 cells into the patient's body.

In the abovementioned method for the treatment and prevention, the step of washing the Th1 cells before returning the cells into the patient's body can be added.

The abovementioned method is advantageous in that the tumor cytotoxicity can be induced without directly administering IL-12 to the body. Namely, although IL-12, a kind of cytokine, has been reported to have an antitumor effect (Takashi Nishimura, Th1/Th2 cells and antitumor immunity—Which cell, Th1 or Th2, is effective for cancer treatment? Igaku no Ayumi, 2000, 194:1230-1236), it is preferable to avoid direct administration of IL-12 to the body from the view point of side effects such as hepatic disorders. According to a method of the present invention, patient's T cells are taken out of the body by drawing blood (using a continuous centrifugation device for separating mononuclear cells), the expression of IL-12 receptors on the cells is stimulated by a specific antigen, and after the action with IL-12, the target cells are washed and returned into the body, which makes it possible to expect therapeutically useful treatment while maintaining safety better than the direct administration of a cytokine.

According to the present invention, there are provided a pharmaceutical composition for the treatment and prevention of uterine cancer comprising a CD4-positive Th2 cell according to the present invention, and use of a CD4-positive Th2 cell according to the present invention for producing a pharmaceutical for use in the treatment and prevention of uterine cancer.

Also, according to the present invention, there is provided a method for treating and preventing uterine cancer, comprising the steps of culturing peripheral blood mononuclear cells isolated from the blood of a uterine cancer patient or a person suspected to be a uterine cancer patient in a medium comprising an epitope according to the present invention, recovering CD4-positive Th2 cells from the culture, and then returning the CD4-positive Th2 cells into the patient's body.

In the abovementioned method for the treatment and prevention, the step of washing Th2 cells before returning the cells into the patient's body can be added.

The abovementioned method is considered to be particularly effective for the early treatment of uterine cancer. According to Example 4, it was revealed that in cases of CIN1 and CIN2 in which the development of lesions was arrested in spite of HPV infection, the frequency of Th2 cells specific to cancer antigens was high, exhibiting a frequency 4 to 36 times higher than that in cases of progressive cancer. Namely, a reverse correlation was recognized between the frequency of Th2 cells and the progress of lesions. Accordingly, it is considered to be effective to artificially increase such T cells for the treatment of uterine cancer.

The step of returning obtained Th1 cells or Th2 cells into the patient's body can be carried out by suspending the cells in a physiological saline solution, Ringer's solution, or the like, and injecting the suspension by continuous intravenous injection, intravenous injection, intracutaneous injection, subcutaneous injection, lymph node injection, or local injection to foci. If necessary, the cells can be administered to the patient with pharmaceutically acceptable additives for pharmaceutical use.

### EXAMPLES

The following examples were carried out using lesion tissues from anonymously treated 125 female patients who visited the department of gynecology in the General Medical Center of Saitama Medical School, requested a cervical cancer examination, gave their written consent to the cooperation with this research and were found to have deformed lesions in the cervix as a result of pathological diagnosis. Further, of the abovementioned 125 patients, with further consent to provide their peripheral blood, lymphocytes from 105 patients and T cells from 12 patients were used in the following Examples.

### Example 1

### DNA Detection and HPV Typing

The detection and typing of HPV is a preliminary study for the selection of cases caused by infection with human papillomavirus (HPV). The study was carried out using a method previously reported (Nakagawa S, Yoshikawa H, Onda T, Kawana T, Iwamoto A, Taketani Y. Type of human papillomavirus is related to clinical features of cervical carcinoma. Cancer 1996, 78:1935-1941). Namely, DNA extracted from a tissue was amplified using primers, which were designed based on the common sequence of L1 regions of HPV, L1C1: CGTAAACGTTTTCCCTATTTTTTT (SEQ ID NO: 2), L1C2: TACCCTAAATACTCTGTATTG (SEQ ID NO: 3), and L1C2M: TACCCTAAATACCCTATATTG (SEQ ID NO: 4), after which the DNA was digested and cleaved using DdleI and RsaI. This DNA was subjected to electrophoresis on 4% agarose gel and the type of HPV was determined based on the length of the DNA fragment. DNAs for which no PCR amplification was recognized were considered to be HPV negative.

As a result, HPV DNA was detected in 105 out of 125 lesion tissues examined. Among them, 65 cases were HPV 16 type, which is known to be most highly involved in the etiology of cervical cancer.

### Example 2

### HLA Analysis (Human MHC Class I and Class II Alleles

As for the correlation between cancerous and precancerous lesions of the cervix and MHC class II, there have been several analytical reports on HLA allelle frequency in Western and Japanese patients having cancerous and precancerous lesions of the cervix. These reports reveal that there is a correlation between the progress of the lesions and the frequency of specific HLA alleles, in particular an MHC class II allele. Thus, presumably, when infected with HPV, the MHC dependent-cellular immune reaction through antigen-presenting cells and T cells is different in different alleles in terms of HPV elimination, which consequently affects the frequency of canceration (Nawa A, Nishiyama Y, Kobayashi T, Wakahara Y, Okamoto T, Kikkawa F. Association of human leukocyte antigen-B1*03 with cervical cancer in Japanese women aged 35 years and younger. Cancer. 1995, 75:518-521; Odunsi K, Terry G, Ho L, Bell J, Cuzick, Ganesan TS. Susceptibility to human papillomavirus-associated cervical intra-epithelial neoplasia is determined by specific HIA DR-DQ alleles. Int J Cancer 1996, 67:595-602; Montoya L, Saiz I, Rey G, Vela F, Clerici-Larradet N. Cervical carcinoma: human papillomavirus infection and HLA-associated risk factors in the Spanish population. Eur J Immunogenetics 1998, 25: 329-337; Allen M, Kalantsri M, Ylitalo N, Pettersson B, Hagmar B, Scheibenphlug L, et al. HLA DQ-DR haplotypes and susceptibility to cervical carcinoma: indications of increased risk for development of cervical carcinoma in individuals infected with HPV 18. Tissue Antigens 1996, 48:32-37).

HLA analysis in this Example is a test to preliminarily confirm the correlation between the type of HLA and the cancer cases of 65 patients specified in Example 1. The test was carried out according to the method previously reported (Kishimoto T, Matsuki K, Islam M M, Hirata R, Maeda H. Unique associations between HLA-B and HLA-DRB1*04 gene variations in Japanese. Tissue Antigens 1993, 42:497-501). Namely, DNAs derived from peripheral blood mononuclear cells of 64 HPV16-positive cases were determined for HLA (human leucocyte antigens) (MHC) class I A and B, and class II DRB1 and DQB1 using HLA test methods considered to be currently standard methods, namely a PCR-reverse SSO method and a PCR-SSCP method. Results are shown in Table 1.

TABLE 1
class II allele frequency (%)

| | Total | HPV 16+ | | |
|---|---|---|---|---|
| HLA allele | patients | CIN 1 & 2 | CIN 3, CIS, IC | Healthy subjects |
| DRB1* 0405 | 21.0 | 20.0 | 14.8 | 28.3 |
| DRB1* 0901 | 40.0 | 30.0 | 55.6 | 29.7 |
| DRB1* 1302 | 5.7 | 0 | 3.7 | 14.5 |
| DQB1* 0302 | 15.2 | 10.0 | 18.5 | 15.9 |
| DQB1* 03032 | 39.0 | 40.0 | 55.6 | 28.3 |
| DQB1* 0601 | 48.6 | 40.0 | 48.2 | 36.2 |

Table 1 shows results of the analysis of HLA allele frequency in the 65 cases. The CIN1 and CIN2 are case groups having cervical lesions with a lower level of deformity, and the CIN3, CIS, and IC are case groups having progressive lesions with a precancerous state, an intradermal carcinoma and a progressive carcinoma, respectively. The frequency for HLA DRB1*0405 allele was lower in the group with progressive lesions than in the healthy subjects, the frequency for HLA DRB1*0901 was high in the group with progressive lesions, and the frequency for HLA DRB1*1302 was low in both groups. Further, the frequency for HLA DQB1 fluctuated due to unbalance linked with DRB1. Further, no significant difference was observed for the class I allele frequency.

### Example 3

### Confirmation of the Presence of Humoral Immunity Against Cancer Antigens

In order to analyze whether specific antibodies against cancer antigens are present in the serum of cases, the reaction between a soluble protein fraction of small cell lung cancer cell line (CRL-2078, American Type Culture Collection) transformed with a plasmid containing the HPV E6/E7 genes and the serum of cases was studied by the Western Blot method. This method is a standard method for biochemical analysis in which an antigen-antibody reaction takes place on a nitrocellulose membrane. This analysis was carried out using a commercially available protein detector, Western Blot Kit (Kirkegaard & Perry Laboratories, Gaitherburg, Md., USA). Results are shown in Table 2.

TABLE 2
Detection of antibodies against E6/E7 proteins

| | | Number of positive | |
|---|---|---|---|
| Lesions | Number of subjects | subjects | Frequency (%) |
| IC | 5 | 2 | 40.0 |
| CIS | 5 | 1 | 20.0 |
| CIN3 | 7 | 2 | 28.6 |
| CIN2 | 7 | 1 | 14.3 |
| CIN1 | 4 | 4 | 100.0 |
| Total | 28 | 10 | Average 35.7% |

As a result, in patients with various levels of progression, positive reaction with E6/E7 proteins was observed and thus the presence of antibodies was confirmed (Table 2).

### Example 4

### Detection of Antigen-specific T Cell

Of the amino acid sequence (SEQ ID NO: 5) coding for an E7 protein, 12 kinds of candidate sequences having a possibility to bind to MHC class II DRB1*04 or DRB1*09 that was suspected to be related to the present disease from the result of Example 2 were prepared using synthesized amino acids (see Table 3 and Table 4).

TABLE 3
MHC class II binding motifs

| MHC class II molecules | Consensus motifs |
|---|---|
| HLA-DR1 | K/H/R - - - - Q/N/T/Y ---F/L/I/N |
| HLA-DR1/*04 | W/L/Y/F- - L/I/M - - - - V/L |
| HLA-DR2/DR2 | I/L/V - - - - - - - - - H/K/R |
| HLA-DR3/DRw52 | F/I/L/V/Y - - D/N/Q/T |
| HLA-DR4 | F/L/V - - - - - - - N/Q/S/T |
| HLA-DR7 | F/I/L/V/Y - - - - N/S/T |
| HLA-DR8 | F/I/L/V/Y - - - H/K/R |
| HLA-DRB1*09 | W/F/L - - S/A/V/F |

TABLE 4
Polypetides synthesized from motifs in HPV16 E7

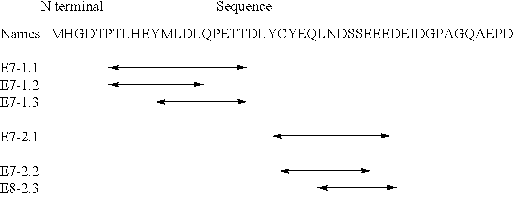

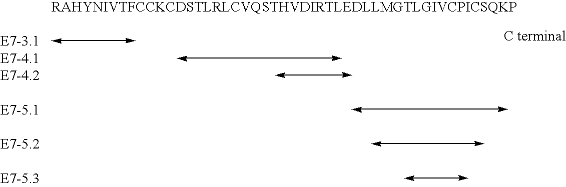

They were cultured with peripheral blood mononuclear cells of patients for 2 days and then IL-4 and CD4 on the surface of T cells were analyzed by the flow cytometry method. A T cell that is positive for IL-4 and CD4 and recognizes the peptides can be identified as a Th2-type antigen specific T cell. Analysis to detect this T cell was carried out by the following procedure.

(1) Preparation of Cells

The venous blood (20 ml) of a subject was drawn into a tube with 2 ml of 10% heparin added. Mononuclear cells were separated from this blood by specific gravity centrifugation. These cells were suspended in physiological saline and the cell number was counted to prepare a suspension containing mononuclear cells of 1.8×10⁷ or more in total. Next, the mononuclear cell suspension was replaced by an RPM 11640 liquid culture medium (Iwaki Glass) supplemented with 10% calf serum (GIBCO, BRL, NY, USA), 0.1 g/l streptomycin (Meiji Seika), and 100,000 units/i penicillin G (Meiji Seika) adjusting the cell concentration to 1×10⁶/ml. These cells were inoculated onto a 12-well plastic culture plate. For comparison, 2 kinds of plates were prepared to independently detect IFNγ produced by Th1-type T cells and IL-4 produced by Th2-type T cells. Further, each plate was subdivided for with and without an antigenic peptide and with and without addition of IL-2 or IL-12, and cultured at 37° C. for 48 hours in a cell culture device under an atmosphere of 5% CO₂. A negative control was a group with no an antigenic peptide added and a positive control was a group with addition of an HA antigen (influenza virus hemagglutinin peptide: HA307-319, PKYVKQNTLKLAT, custom synthesized peptide; Sawaday Technology, Tokyo) (Nobusawa E, Aoyama T, Kato H, Suzuki Y, Tateno Y, Nakajima K. Comparison of complete amino acid sequences and receptor-binding properties among 13 serotypes of hemagglutinins of influenza A viruses. Virology 1991,182:475-485). Further, the concentration of added recombinant IL-2 (Dainippon Seiyaku, Tokyo) was 10 μl/ml, the concentrations of the tested peptide were 10, 20 and 50 nmol, and the amount of recombinant IL-12 (Dainippon Seiyaku, Tokyo) was 0.2 ng/ml/well.

(2) Preparation of Antigens

For antigenic peptides, chemical synthesis was performed to prepare amino acid sequences consisting of 9 to 20 amino acid residues of all patterns among the amino acid sequences of human HPV16 E6 and E7 proteins, which were presumed to contain at least a part of agretope exhibited in so-called MHC class II consensus motifs (see Table 3) (Hammer J, Valsasnini P, Tolba K, Bolin D, Higelin J, Takacs B, Sinigaglia F. Promiscus and allele-specific anchors in HLA-DR-binding peptides. Cell 1993, 74:197-203; Fujisao S. Matsushita S, Nishi T, Nishimura Y. Identification of HLA-DR9(DRB1*0901)-binding peptide motifs using a phage fUSE5 random peptide library. Human Immunol. 1996, 45:131-136) which were considered to have a high affinity with HLA DRB1*0405 molecule and HLA DRB1*0901 molecule that exhibited lower HLA DRB1 allele frequency in the cases than in the healthy subjects in the present study (see Table 3). The peptides were synthesized by chemically bonding individual amino acid reagents using an automatic synthesizer. Optional amino acid sequences were entrusted in advance and synthesized products based on these sequences were used (custom-synthesized peptides, Sawaday Technology, Tokyo).

(3) Detection of IFNγ, IL-4 Cytokine and CD4

For detecting a cytokine, an analysis was carried out by flow cytometry using a monoclonal antibody, which recognizes the cytokine, modified with a fluorescent substance such as phycoerythrin (PE) and fluorescein isothiocyanate (FITC). A commercial detection kit and a monoclonal antibody were used in combination so that the antibody can penetrate the cell membrane to reach inside the cell since the antibody cannot cross the cell membrane to reach inside the living cell. Specifically, a Cytofix/Cytoperm Kit (Pharmingen, San Diego, Calif., USA) and phycoerythrin-conjugated mouse anti-human IL-4 monoclonal antibody: D4-8 (Pharmingen), phycoerythrin-conjugated mouse anti-human IFN-γ monoclonal antibody: D4-8 (Pharmingen), FITC-conjugated mouse anti-human CD-4 monoclonal antibody (Miltenyi Biotec, Glandbach, Germany), and FITC-conjugated mouse anti-human CD-8 monoclonal antibody (Miltenyi Biotec, Glandbach, Germany) were used for staining and positive cells were detected using a flow cytometry device, FACScan (Becton Dickinson, San Jose, Calif., USA).

More specifically, 2 hours before the end of culture, 0.7 μl/ml/well of Brefeldin A was added to stop the protein production in the Golgi apparatus and cells stimulated with an antigen were treated at 37° C. for 2 hours. The culture plate was placed on ice and PBS at 4° C. was added to stop the reaction. The cells were washed with PBS into a 15 ml tube at 4° C., and the supernatant was removed by centrifugation at 1600 rpm for 10 minutes. Next, in order to stain cell surface antigens, 10 μl of FITC-labeled mouse anti-human CD4 or CD8 monoclonal antibody was added and reacted at 4° C. for 30 minutes. Then, an appropriate amount of 0.5% BSA/PBS was added at 4° C. to loosen the cells, 0.5% BSA/PBS was further added at 4° C. and then centrifugation was carried out at 4° C. for 10 minutes at 1600 rpm to remove the supernatant.

Next, cell immobilization and cell membrane permeation were carried out. Cells were suspended in 250 μl of a cytofix/cytoperm solution and reacted at 4° C. for 20 minutes. To this cell suspension, 10 μl of the abovementioned IFNγ or IL-4 antibody was added and reacted at 4° C. for 30 minutes. Then, an appropriate amount of a 0.5% BSA/PBS solution was added at 4° C. to loosen the cells, 0.5% BSA/PBS was further added at 4° C., and then centrifugation was carried out at 4° C. for 10 minutes at 1600 rpm to remove the supernatant, after which the cells were washed, suspended in PBS and analyzed using a FACscan apparatus (Kazuko Shibuya, Detection of Intracellular Cytokines, Flow Cytometry with Complete Control, compiled by Hiromitsu Nakauchi, pp 75-85, 1999, Shujunsha, Tokyo). Cells were irradiated with a laser beam to catch excited fluorescence and the presence of the cells binding the target antibody was indicated by calculation on a computer. The positive cell frequency in comparison with the control was calculated.

(4) Identification of T cell Epitopes

IL-4- and CD4-positive T cell frequency was analyzed for 12 cases mainly having HLA DRB1*0901 or DRB1*0405. When individual polypeptides were compared, a highly positive reaction was observed with E7-4.1: HPV E7 61-80: DSTLRLCVQSTHVDIRTLE. Further, positive cells were detected, although at a low frequency, with E7-5.1: HPV E7 82-95: LLMGTLGIVCPICS. However, since no reaction was found with other peptides, it was proved that an epitope site where a T cell recognizes and an agretope which binds to HLA DRB1*0901 or DRB1*0405 were built-in in the E7-4.1 DSTLRLCVQSTHVDIRTLE.

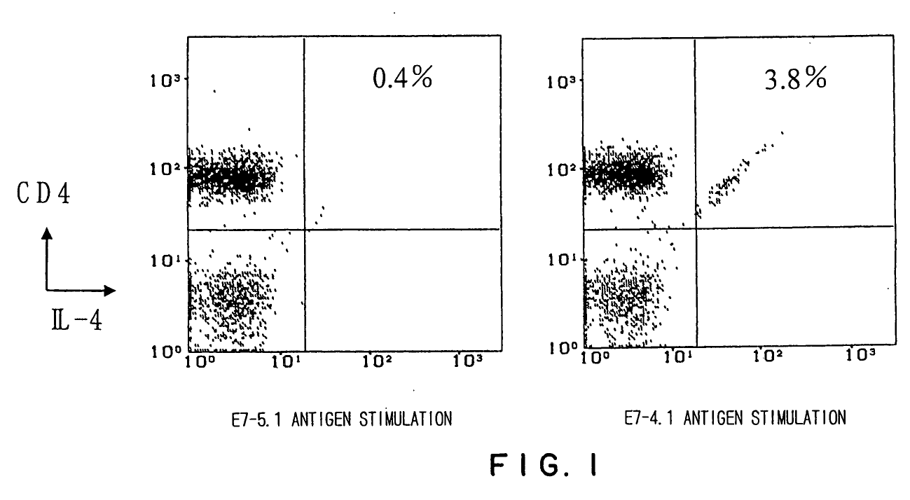

The positive T cell frequency upon stimulation with E7-4.1 was 0.3% to 3.6% of the total peripheral blood mononuclear cells (Table 5: the whole result, FIG. 1: a typical example).

TABLE 5
Positive frequency of Th2-type T cells specific to E7-4.1 peptide, and lesions
and HLA of cases

| | | | | | | | | | Peptide reaction | | | | |
|---|---|---|---|---|---|---|---|---|---|---|---|---|---|
| | | | | | | | | | (positive T cell frequency(%)) | | | | |
| Lesions | class I | | | | HLA | | DRB1 | DQB1 | E7-1.1 | E7-2.1 | E7-3.1 | E7-4.1 | E7-5.1 |
| IC | A2 | A26 | B55 | B60 | | | 0803:0901 | 0601:03032 | 0 | 0 | 0 | 0.9 | 0 |
| IC | A11 | A24 | B54 | | C1 | | 0405:0802 | 0302:0401 | 0 | 0 | 0 | 0.3 | 0 |
| IC | A24 | A33 | B44 | B39 | C7 | | 0803:1302 | 0601:0604 | 0 | 0 | 0 | 0 | 0 |
| CIS | A2 | A24 | B61 | B62 | C1 | C10 | 0803:0901 | 0601:03032 | 0 | 0 | 0 | 0.3 | 0 |
| CIS | A24 | A26 | B61 | B62 | C10 | | 1502:0901 | 0601:03032 | 0 | 0 | 0 | 0.2 | 0.2 |
| CIS | A24 | | B51 | B52 | | | 1502:0901 | 0601:03032 | 0 | 0 | 0 | 0 | 0 |
| CIN2 | A2 | A24 | B51 | B7 | C7 | | 0101:1501 | 0501:0602 | 0 | 0 | 0 | 1.2 | 0.8 |
| CIN2 | A24 | | B52 | B54 | C1 | | 1502:0405 | 0601:0401 | 0 | 0 | 0 | 0.1 | 0 |
| CIN1 | A24 | A31 | B7 | | C7 | | 0101:0901 | 0501:03032 | 0 | 0 | 0 | 1.3 | 0 |
| CIN1 | A24 | A31 | B56 | B7 | C7 | C4 | 0101:0901 | 0501:03032 | 0 | 0 | 0 | 1.1 | 0 |
| CIN1 | A2 | A26 | B35 | B46 | C1 | C9 | 0403:1405 | 05031:0302 | 0 | 0 | 0 | 3.6 | 0.4 |
| Normal | A2 | A33 | B44 | B7 | | | 0405:1302 | 0604:0401 | 0 | 0 | 0 | 0 | 0 |
| Normal | A24 | A31 | B52 | B39 | C7 | | 1502:0803 | 0604 | 0 | 0 | 0 | 0 | 0 |
| Normal | A24 | A33 | B52 | B44 | | | 1502:1302 | 0601:0604 | 0 | 0 | 0 | 0 | 0 |

Note:
0 (zero) represents less than 0.1%

Regarding the relation between the level of progress in lesions and the positive cell frequency, the positive frequency was less than 0.1% in healthy females, and as high as 1.0% or higher in 3 out of 3 cases of CIN1 and 1.2% in 1 out of 2 cases of CIN2 (Table 6).

TABLE 6
Relation between positive frequency of Th2-type T cells
specific to E7-4.1 peptide and lesions of cases

| | Number of | Less than | 0.1% to less | 1.0% | Positive T cell |
|---|---|---|---|---|---|
| Lesions | subjects | 0.1% | than 1.0% | or move | frequency (%) |
| IC | 3 | 1 | 1 | 1 | 0˜0.9 |
| CIS | 3 | 1 | 2 | 0 | 0˜0.3 |
| CIN2 | 2 | 0 | 1 | 1 | 0.1˜1.2 |
| CIN1 | 3 | 0 | 0 | 3 | 1.1˜3.6 |
| Normal | 3 | 3 | 0 | 0 | less than 0.1 |
| Total | 14 | 5 | 4 | 5 | |

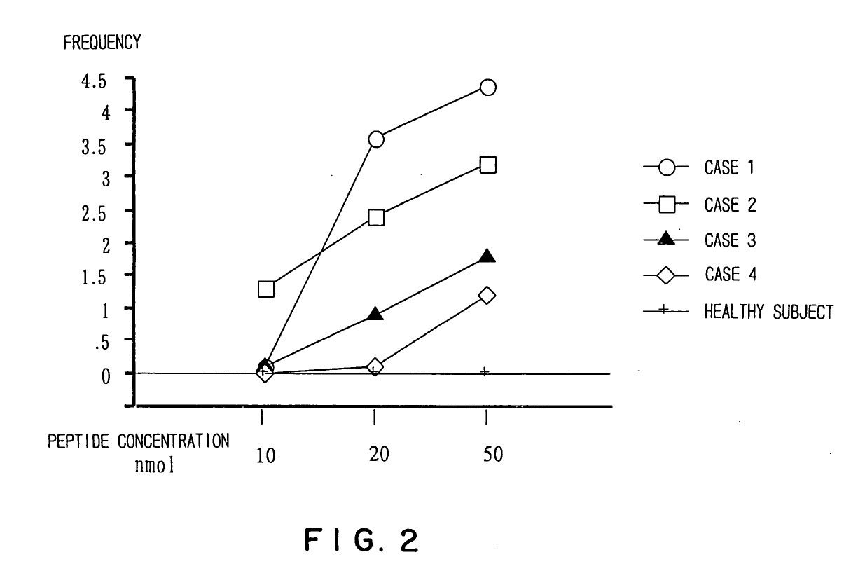

Further, the reactivity of the positive cases showed a dependency on the concentration of E7-4.1 peptide antigen (FIG. 2).

In an analysis with healthy subjects using HA of influenza antigen, IL-4-positive CD4-positive T cells of subjects already infected with influenza were detected by peptide stimulation at a frequency of 0.1-0.5% (for reference, the frequency of CD4-positive IFNγ-positive T cells was 0.7-3.0%). The background value of 0.1-0.3% of positive T cell frequency without stimulation was deducted in advance from all the values of these analytical results. Further, positive control cells attached to the kit were used in the analysis to maintain the reproducibility of the measurements.

### Example 5

### Induction of Th1-type T Cells

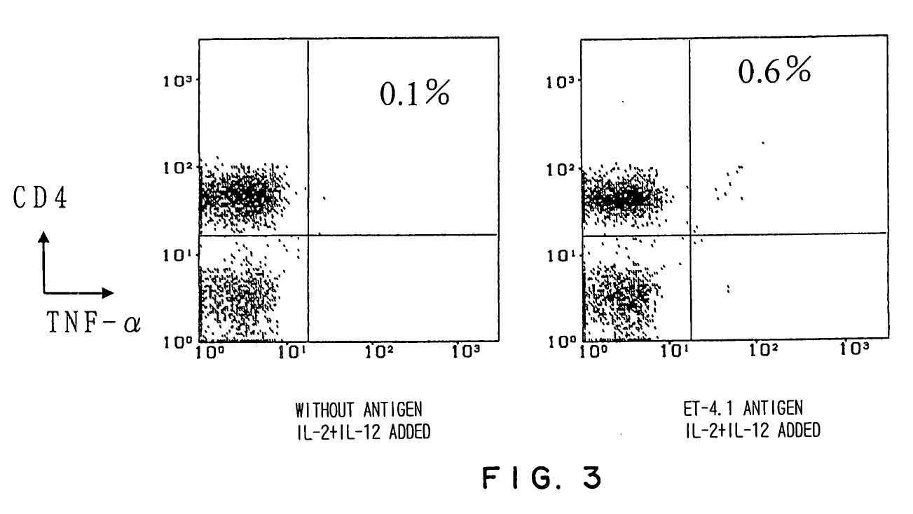

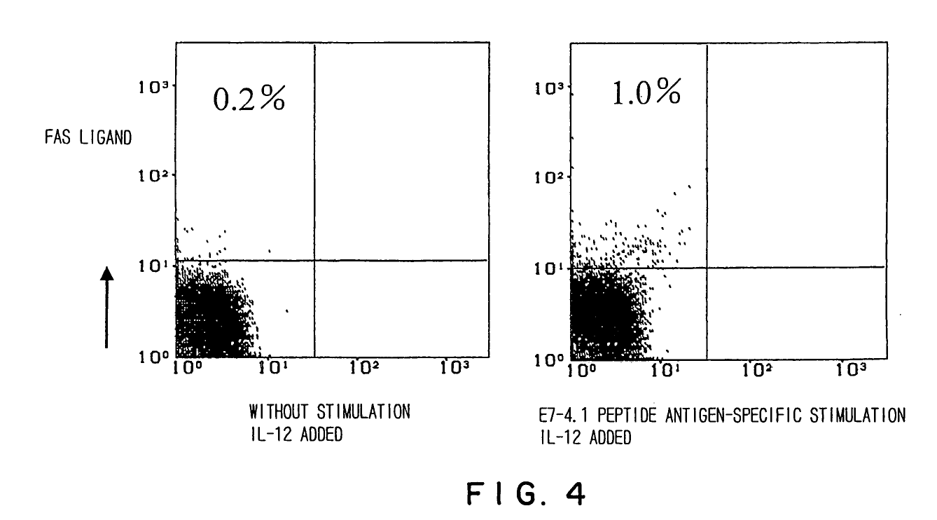

The growth of D4-positive T cells producing IFNγ, namely Th1-type T cells, was recognized when IL-12 was added in culturing the abovementioned human papillomavirus polypeptide E7-4.1: DSTLRLCVQSTHVDIRTLE and patient's mononuclear cells (FIG. 3 and FIG. 4). These Th1-type T cells presumably recognize similar epitopes as the abovementioned Th2-type T cells. Further, Th1-type T cells produce TNFα (tumor necrosis factor alpha) more strongly than Th2 cells so that they can be accumulated antigen-specifically onto cancer cells to exhibit cytotoxic activity, which may suppress tumor cells. Actually the TNFα and a cytotoxic factor Fas ligand were detected in the cells obtained. FIG. 3 shows the two-color flow cytometry of CD4 and TNFα and FIG. 4 shows the single color flow cytometry of Fas ligand.

1. A peptide comprising the following amino acid sequence:
(a) the amino acid sequence of SEQ ID NO: 1, or
(b) a modified amino acid sequence of the amino acid sequence of SEQ ID NO: 1 that has one or more modifications selected from the group consisting of a substitution, a deletion, an addition and an insertion and can activate CD4-positive T cells specific to cancerous and precancerous lesions of the uterus.

2. A CD4-positive Th1 cell specific to cancerous and precancerous lesions of the uterus, which is obtainable by culturing peripheral blood mononuclear cells in a medium comprising the peptide of claim 1 and Il-12.

3. The CD4-positive Th1 cell according to claim 2, which can induce tumor cytotoxicity against cancerous and precancerous lesions of the uterus.

4. The CD4-positive Th1 cell according to claim 2, wherein the cancerous and precancerous lesions of the uterus are caused by papillomaviruses.

5. The CD4-positive Th1 cell according to claim 2, 3 or 4, wherein the peripheral blood mononuclear cells are isolated from the blood of a uterine cancer patient or a person suspected to be a uterine cancer patient.

6. A CD4-positive Th1 cell that can induce tumor cytotoxicity against cancerous and precancerous lesions of the uterus expressing a human papillomavirus 16 E7 protein, which is obtainable by culturing peripheral blood mononuclear cells isolated from the blood of a uterine cancer patient or a person suspected to be a uterine cancer patient in a medium comprising a peptide comprising the amino acid sequence of SEQ ID NO: 1 and IL-12.

7. A CD4-positive Th2 cell specific to cancerous and precancerous lesions of the uterus, which is obtainable by culturing peripheral blood mononuclear cells in a medium comprising the peptide of claim 1.

8. The CD4-positive Th2 cell according to claim 7, which can induce the production of an antibody specific to cancerous and precancerous lesions of the uterus.

9. The CD4-positive Th2 cell according to claim 7, wherein the cancerous and precancerous lesions of the uterus are caused by papillomavirus.

10. The CD4-positive Th2 cell according to claim 7, 8 or 9, wherein the peripheral blood mononuclear cells are isolated from the blood of a uterine cancer patient or a person suspected to be a uterine cancer patient.

11. A CD4-positive Th2 cell that can induce the production of an antibody specific to cancerous and precancerous lesions of the uterus expressing a human papillomavirus 16 E7 protein, which is obtainable by culturing peripheral blood mononuclear cells isolated from the blood of a uterine cancer patient or a person suspected to be a uterine cancer patient in a medium comprising a peptide consisting of the amino acid sequence of SEQ ID NO: 1.

12. A pharmaceutical composition for treating and preventing uterine cancer, comprising the CD4-positive Th1 cell of any one of claims 2 to 6 and/or the CD4-positive Th2 cell of any one of claims 7 to 11.

13. The pharmaceutical composition according to claim 12 for use in tumor therapy.

14. Use of the CD4-positive Th1 cell of any one of claims 2 to 6 and/or the CD4-positive Th2 cell of any one of claims 7 to 11 for the production of a pharmaceutical for use in the treatment and prevention of uterine cancer.

15. A method for treating and preventing uterine cancer, comprising the steps of culturing peripheral blood mononuclear cells isolated from the blood of a uterine cancer patient or a person suspected to be a uterine cancer patient in a medium comprising the peptide of claim 1 and IL-12, collecting CD4-positive Th1 cells from the culture, and then returning the CD4-positive Th1 cells into the patient's body.

16. A method for treating and preventing uterine cancer, comprising the steps of culturing peripheral blood mononuclear cells isolated from the blood of a uterine cancer patient or a person suspected to be a uterine cancer patient in a medium comprising the peptide of claim 1, collecting CD4-positive Th2 cells from the culture, and then returning the CD4-positive Th2 cells into the patient's body.

17. A method of producing CD4-positive Th1 cells that can induce tumor cytotoxicity against cancerous and precancerous lesions of the uterus, comprising the steps of culturing peripheral blood mononuclear cells isolated from the blood of a uterine cancer patient or a person suspected to be a uterine cancer patient in a medium comprising the peptide of claim 1 and IL-12 and then recovering CD4-positive Th1 cells.

18. A method of producing CD4-positive Th2 cells that can induce the production of an antibody specific to cancerous and precancerous lesions of the uterus, comprising the steps of culturing peripheral blood mononuclear cells isolated from the blood of a uterine cancer patient or a person suspected to be a uterine cancer patient in a medium comprising the peptide of claim 1 and then recovering CD4-positive Th2 cells.

19. An agent for activating the ability of a CD4-positive Th1 cell to induce tumor cytotoxicity specific to cancerous and precancerous lesions of the uterus, comprising the peptide of claim 1.

20. The activating agent according to claim 19, which is used in combination with IL-12.

21. An agent for activating the ability of a CD4-positive Th2 cell to induce the production of an antibody specific to cancerous and precancerous lesions of the uterus, comprising the peptide of claim 1.

22. Use of the peptide of claim 1 for the production of an agent for activating the ability of a CD4-positive Th1 cell to induce tumor cytotoxicity specific to cancerous and precancerous lesions of the uterus.

23. Use of the peptide of claim 1 for the production of an agent for activating the ability of a CD4-positive Th2 cell to induce the production of an antibody specific to cancerous and precancerous lesions of the uterus.

24. A method of activating the ability of a CD4-positive Th1 cell to induce tumor cytotoxicity specific to cancerous and precancerous lesions of the uterus, comprising the step of bringing the peptide of claim 1 into contact with the CD4-positive Th1 cell in the presence of IL-12.

25. A method of activating the ability of a CD4-positive Th2 cell to induce the production of an antibody specific to cancerous and precancerous lesions of the uterus, comprising the step of bringing the peptide of claim 1 into contact with the CD4-positive Th2 cell.
